(19) United States
(12) Patent Application Publication
(10) Pub. No.: US 2024/0156331 A1
(43) Pub. Date: May 16, 2024

(54) ENDOSCOPIC DEVICE

(71) Applicant: ALTEK BIOTECHNOLOGY CORPORATION, Hsinchu City (TW)

(72) Inventors: Liang-Yi Li, Hsinchu City (TW); Chun-Wei Liu, Hsinchu City (TW)

(21) Appl. No.: 18/506,148

(22) Filed: Nov. 10, 2023

Related U.S. Application Data
(60) Provisional application No. 63/424,941, filed on Nov. 13, 2022.

(51) Int. Cl. A61B 1/00; A61B 1/05; A61B 1/06; A61B 1/07; A61B 1/12; H04N 23/50; H04N 23/55; H04N 23/56

(52) U.S. Cl. CPC A61B 1/00096; A61B 1/05; A61B 1/0676; A61B 1/07; A61B 1/128; H04N 23/55; H04N 23/555; H04N 23/56

(57) ABSTRACT

An endoscopic device is provided and includes an insertion tube, an imaging assembly, a light guiding component and a light emitting component. The imaging assembly includes a lens set and an image sensor. The lens set is located adjacent to a distal end of the insertion tube. The image sensor is located inside the insertion tube and engaged with the lens set. The light guiding component is located adjacent to the distal end of the insertion tube and beside the lens set. The light guiding component includes an outer end adjacent to the distal end of the insertion tube and an inner end opposite to the outer end. A length of the light guiding component is greater than a length of the imaging assembly. The light emitting component is located inside the insertion tube and adjacent to the inner end of the light guiding component.

### CROSS REFERENCE TO RELATED APPLICATIONS

This application claims the benefit of U.S. Provisional Application No. 63/424,941, filed on Nov. 13, 2022. The content of the application is incorporated herein by reference.

### BACKGROUND OF THE INVENTION

### 1. Field of the Invention

The present invention relates to an endoscopic device, and more specifically, to an endoscopic device with a compact size and an easy assembling process.

### 2. Description of the Prior Art

An endoscopy is a medical procedure in which an endoscope is inserted into a patient's body to allow an inspection of an interior of the patient's body. The endoscopy has gained broad acceptance because it only needs a small incision or natural orifice for insertion of the endoscope. However, the conventional endoscope is bulk, such that a size of the incision cannot be further reduced in order for insertion of the endoscope. Besides, the conventional endoscope has a complicated assembling process. Therefore, an improvement is required.

### SUMMARY OF THE INVENTION

It is an objective of the present invention to provide an endoscopic device with a compact size and an easy assembling process for solving the aforementioned problems.

In order to achieve the aforementioned objective, the present invention discloses an endoscopic device. The endoscopic device includes an insertion tube, an imaging assembly, a light guiding component and a light emitting component. The insertion tube includes a proximal end and a distal end opposite to the proximal end. The imaging assembly includes a lens set and an image sensor. The lens set is located adjacent to the distal end of the insertion tube. The image sensor is located inside the insertion tube and engaged with the lens set. The light guiding component is located adjacent to the distal end of the insertion tube and beside the lens set. The light guiding component includes an outer end adjacent to the distal end of the insertion tube and an inner end opposite to the outer end. A length of the light guiding component is greater than a length of the imaging assembly. The light emitting component is located inside the insertion tube and adjacent to the inner end of the light guiding component.

According to an embodiment of the present invention, the light emitting component is located at a side of the image sensor away from the distal end of the insertion tube.

According to an embodiment of the present invention, the inner end of the light guiding component protrudes beyond the image sensor along a length direction of the insertion tube toward the proximal end of the insertion tube.

According to an embodiment of the present invention, the endoscopic device further includes an auxiliary component and a heat dissipating pin. The auxiliary component is located adjacent to the light emitting component. The heat dissipating pin is partially embedded inside the auxiliary component.

According to an embodiment of the present invention, the light emitting component is partially surrounded by the auxiliary component.

According to an embodiment of the present invention, the endoscopic device further includes a holding component detachably engaged with the auxiliary component and for holding the light guiding component or/and the imaging assembly.

According to an embodiment of the present invention, a first auxiliary structure is formed on a first end portion of the auxiliary component adjacent to the proximal end of the insertion tube. At least one second auxiliary structure is formed on a second end portion of the auxiliary component opposite to the first end portion of the auxiliary component. The first auxiliary structure is configured to accommodate a portion of the heat dissipating pin exposed out of the auxiliary component, and the at least one second auxiliary structure is configured to engage with the holding component.

According to an embodiment of the present invention, the holding component includes two holding portions, and the light guiding component and the imaging assembly are at least partially located inside a space defined between the two holding portions.

According to an embodiment of the present invention, the endoscopic device further includes a heat dissipating tube at least partially and coaxially disposed inside the insertion tube.

According to an embodiment of the present invention, a heat dissipating glue is filled in a space between the heat dissipating tube and the heat dissipating pin and/or a space between the heat dissipating tube and the auxiliary component.

According to an embodiment of the present invention, the endoscopic device further includes a heat dissipating tube and an auxiliary component. The heat dissipating tube is at least partially and coaxially disposed inside the insertion tube. The auxiliary component is at least partially disposed between heat dissipating tube and the light emitting component.

According to an embodiment of the present invention, a heat dissipating glue is filled in a space between the heat dissipating tube and the auxiliary component.

According to an embodiment of the present invention, the endoscopic device further includes a first circuit board, a second circuit board and an additional heat dissipating component. The first circuit board is electrically connected to the light emitting component. The second circuit board is electrically connected to the image sensor. The additional heat dissipating component is disposed between the first circuit board and the second circuit board.

According to an embodiment of the present invention, the first circuit board is integrated with the second circuit board or separated from the second circuit board.

In order to achieve the aforementioned objective, the endoscopic device includes an insertion tube, a heat generating component and a heat dissipating tube. The insertion tube includes a proximal end and a distal end opposite to the proximal end. The heat generating component is located adjacent to the distal end of the insertion tube. The heat dissipating tube is at least partially and coaxially disposed inside the insertion tube. The heat dissipating tube is located at a side of the heat generating component away from the distal end of the insertion tube.

According to an embodiment of the present invention, the endoscope device further includes an auxiliary component at least partially disposed between heat dissipating tube and the heat generating component.

According to an embodiment of the present invention, the endoscopic device further includes a heat dissipating pin partially embedded inside the auxiliary component.

According to an embodiment of the present invention, a heat dissipating glue is filled in a space between the heat dissipating tube and the heat dissipating pin and/or a space between the heat dissipating tube and the auxiliary component.

According to an embodiment of the present invention, a first auxiliary structure is formed on a first end portion of the auxiliary component adjacent to the proximal end of the insertion tube, and the first auxiliary structure is configured to accommodate a portion of the heat dissipating pin exposed out of the auxiliary component.

According to an embodiment of the present invention, a heat dissipating glue is filled in a space between the heat dissipating tube and the auxiliary component.

In summary, the endoscopic device of the present invention can not only have a compact size and an easy assembling process, but also have an enhanced illumination and an efficient heat dissipation.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of an endoscopic device according to a first embodiment of the present invention.

FIG. 2A and FIG. 2B are partial internal structural diagrams of the endoscopic device according to the first embodiment of the present invention.

FIG. 3 and FIG. 4 are partial exploded diagrams of the endoscopic device at different views according to the first embodiment of the present invention.

FIG. 5 is a partial internal structural diagram of an endoscopic device according to a second embodiment of the present invention.

FIG. 6 and FIG. 7 are partial exploded diagrams of the endoscopic device at different views according to the second embodiment of the present invention.

FIG. 8 is a partial internal structural diagram of an endoscopic device according to a third embodiment of the present invention.

FIG. 9 and FIG. 10 are partial exploded diagrams of the endoscopic device at different views according to the third embodiment of the present invention.

### DETAILED DESCRIPTION

In the following detailed description of the preferred embodiments, reference is made to the accompanying drawings which form a part hereof, and in which is shown by way of illustration specific embodiments in which the invention may be practiced. In this regard, directional terminology, such as “top”, “bottom”, “front”, “back”, etc., is used with reference to the orientation of the Figure(s) being described. The components of the present invention can be positioned in a number of different orientations. As such, the directional terminology is used for purposes of illustration and is in no way limiting. Accordingly, the drawings and descriptions will be regarded as illustrative in nature and not as restrictive. Also, the term “connect” is intended to mean either an indirect or direct electrical/mechanical connection. Thus, if a first device is connected to a second device, that connection may be through a direct electrical/mechanical connection, or through an indirect electrical/mechanical connection via other devices and connections.

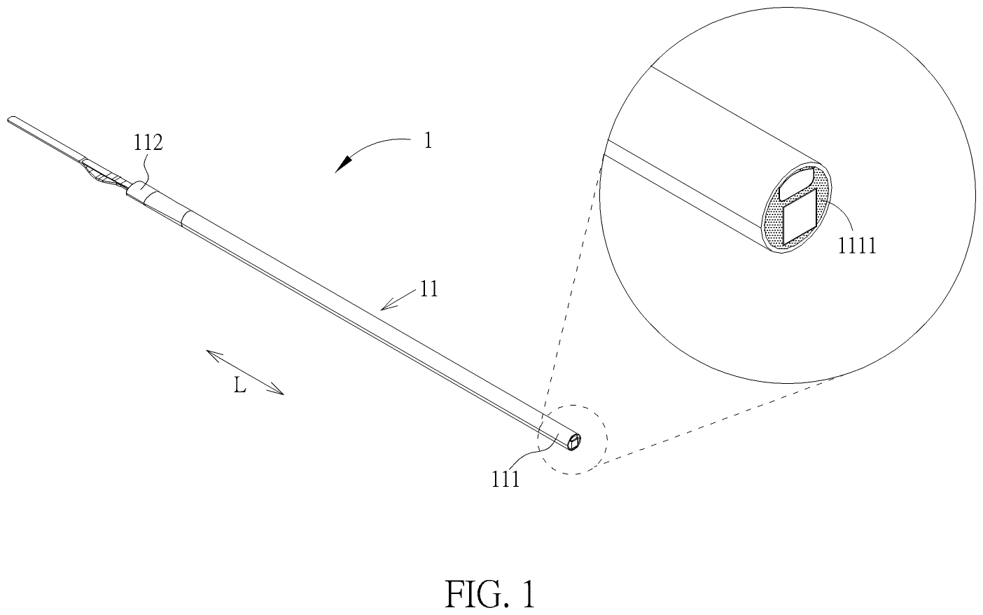

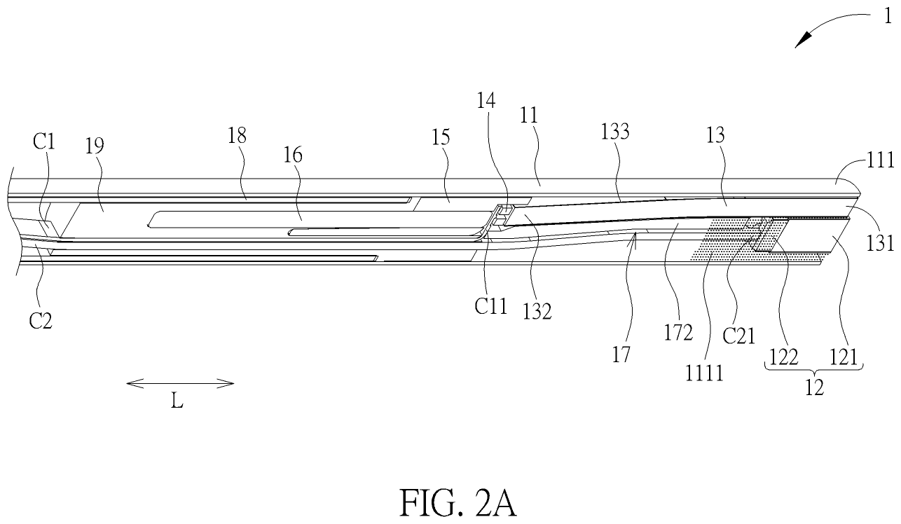

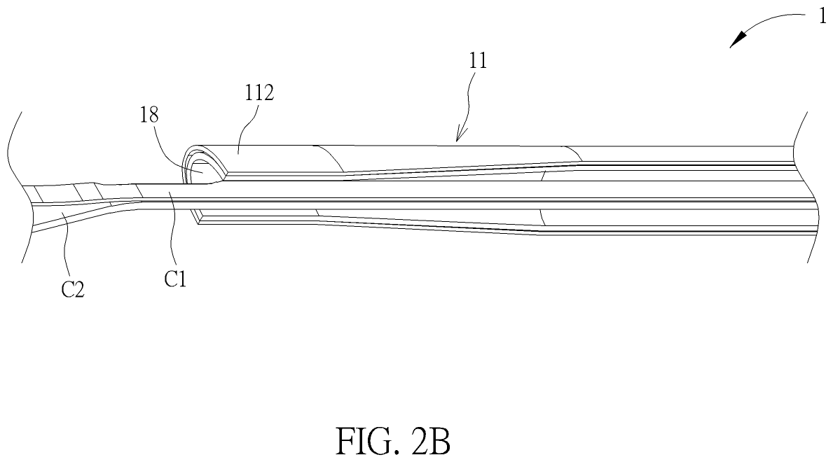

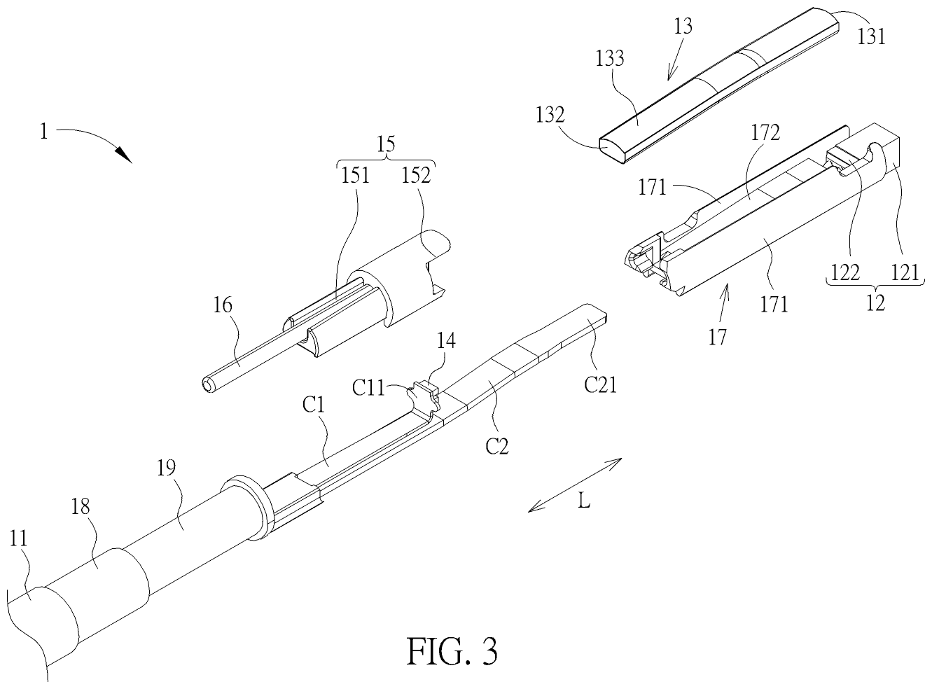

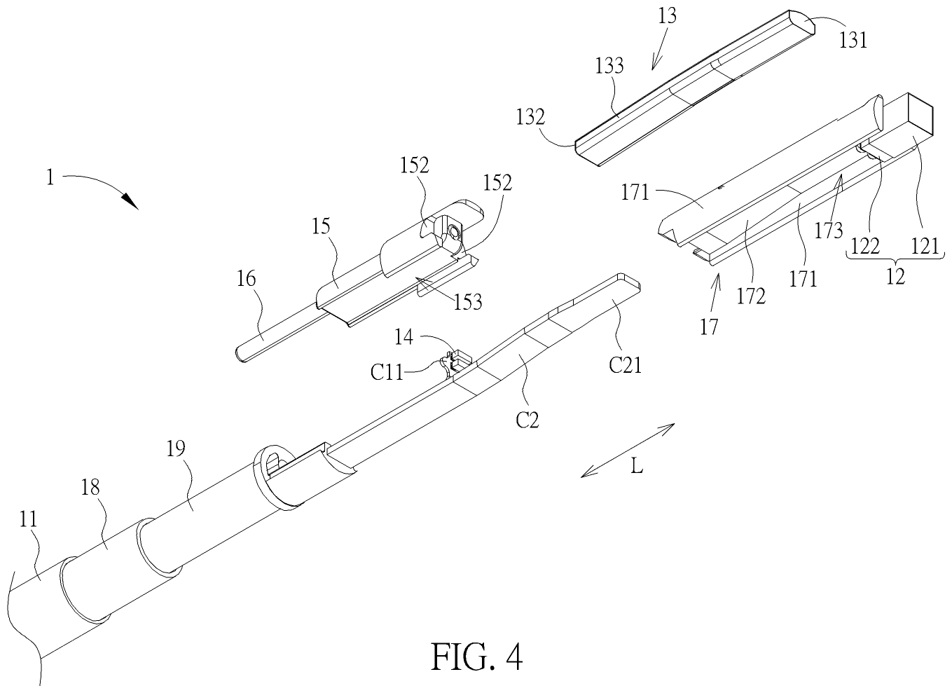

Please refer to FIG. 1 to FIG. 4. FIG. 1 is a schematic diagram of an endoscopic device 1 according to a first embodiment of the present invention. FIG. 2A and FIG. 2B are partial internal structural diagrams of the endoscopic device 1 according to the first embodiment of the present invention. FIG. 3 and FIG. 4 are partial exploded diagrams of the endoscopic device 1 at different views according to the first embodiment of the present invention. As shown in FIG. 1 to FIG. 4, the endoscopic device 1 includes an insertion tube 11, an imaging assembly 12, a light guiding component 13 and a light emitting component 14. The insertion tube 11 includes a distal end 111 and a proximal end 112 opposite to the distal end 111. The proximal end 112 of the insertion tube 11 can be connected to an operating handle, and the distal end 111 of the insertion tube 11 can be inserted into a patient's body. The imaging assembly 12 includes a lens set 121 and an image sensor 122. The lens set 121 is located adjacent to the distal end 111 of the insertion tube 11 for guiding external light to the image sensor 122. The image sensor 122 is located inside the insertion tube 11 and engaged with the lens set 121. The light guiding component 13 is located adjacent to the distal end 111 of the insertion tube 11 and beside the lens set 121 for guiding light emitted from the light emitting component 14 toward an interior of the patient's body. The light guiding component 13 includes an outer end 131 adjacent to the distal end 111 of the insertion tube 11 and an inner end 132 opposite to the outer end 131. A length of the light guiding component 13 is greater than a length of the imaging assembly 12. The light emitting component 14 is located inside the insertion tube 11 and adjacent to the inner end 132 of the light guiding component 13 for emitting light to the light guiding component 13.

Specifically, the light emitting component 14 can be located at a side of the image sensor 122 away from the distal end 111 of the insertion tube 11, and the inner end 132 of the light guiding component 13 can protrude beyond the image sensor 122 along a length direction L of the insertion tube 11 toward the proximal end 112 of the insertion tube 11.

Based on the aforementioned arrangement, a section of the insertion tube 11 for receiving the imaging assembly 12 is located more adjacent to a tip portion of the distal end 111 of the insertion tube 11 than a section of the insertion tube 11 for receiving the light emitting component 14, i.e., the section of the insertion tube 11 for receiving the imaging assembly 12 is in front of the section of the insertion tube 11 for receiving the light emitting component 14, and therefore, the present invention can significantly reduce a predetermined dimension, e.g., a diameter, of the distal end 111 of the insertion tube 11. Besides, the light emitting component 14 can provide a brighter illumination because the light emitting component 14 can have a larger size without interfering the imaging assembly 12.

Preferably, a light emitting surface of the outer end 131 of light guiding component 13 for allowing the light to exit out of the light guiding component 13 can be textured for light diffusion.

Preferably, a light incident surface of the inner end 132 of light guiding component 13 for receiving the light from the light emitting component 14 can be polished for preventing reflection of light emitted from the light emitting component 14 to increase quantity of light into the light guiding component 13.

Preferably, a surface of a lateral portion 133 of the light guiding component 13 can be covered with an optical layer by coating or cladding, and a refractive index of a material of the optical layer is less than a refractive index of a material of the light guiding component 13, so as to prevent refraction of light through the surface of the lateral portion 133 of the light guiding component 13.

Preferably, the light guiding component 13 can be a fiber optical bundle, or alight guiding column made of Polycarbonate (PC), Cyclic olefin copolymer (COC), Polymethyl methacrylate (PMMA) or Liquid silicone rubber (LSR). The refractive index of a material of the optical layer can be from 1.2 to 1.5.

Preferably, the light emitting component 14 can include one or more light emitting diodes (LED).

Preferably, the image sensor 122 can be a Complementary Metal Oxide Semiconductor (CMOS) sensor.

Preferably, the distal end 111 of the insertion tube 11 can be filled with a fluid-proof adhesive 1111 for preventing outside fluid from entering the insertion tube 11 through the distal end 111 of the insertion tube 11. The light emitting surface of the outer end 131 of light guiding component 13, an outer end the lens set 121, a light emitting surface of the light emitting component 14 and the light incident surface of the inner end 132 of light guiding component 13 should not be contaminated by the fluid-proof adhesive 1111.

As shown in FIG. 2A and FIG. 3 to FIG. 4, in this embodiment, the light emitting component 14 can act as a heat generating component because the light emitting component 14 generates heat when emitting light. However, the present invention is not limited to this embodiment. For example, in another embodiment, the heat generating component can be a processor or any other electronic component. In order to dissipate the heat generated by the light emitting component 14 rapidly, the endoscopic device 1 further includes an auxiliary component 15 and a heat dissipating pin 16. The auxiliary component 15 is located adjacent to the light emitting component 14. The heat dissipating pin 16 is partially embedded inside the auxiliary component 15.

Preferably, the heat dissipating pin 16 can be made of copper.

Specifically, the light emitting component 14 is partially surrounded by the auxiliary component 15, so as to effectively dissipate the heat generated by the light emitting component 14 by the auxiliary component 15.

Besides, as shown in FIG. 2A and FIG. 3 to FIG. 4, in this embodiment, the endoscopic device 1 further includes a holding component 17 detachably engaged with the auxiliary component 15 and for holding the light guiding component 13 and the imaging assembly 12.

Specifically, a first auxiliary structure 151 is formed on a first end portion of the auxiliary component 15 adjacent to the proximal end 112 of the insertion tube 11. Two second auxiliary structures 152 are formed on a second end portion of the auxiliary component 15 opposite to the first end portion of the auxiliary component 15. The first auxiliary structure 151 is configured to accommodate a portion of the heat dissipating pin 16 exposed out of the auxiliary component 15, and the two second auxiliary structures 152 are configured to engage with the holding component 17.

Preferably, the first auxiliary structure 151 can be a recess structure, and the second auxiliary structure 152 can be a notch structure.

Furthermore, the holding component 17 includes two holding portions 171 configured to detachably engage with the two second auxiliary structures 152 and a middle portion 172 disposed between the two holding portions 171, and the light guiding component 13 and the imaging assembly 12 are at least partially located inside a space defined between the two holding portions 171.

Understandably, in another embodiment, there can be only one second auxiliary structure configured to engage with the holding component, and the holding component can be only configured to provide a receiving recess structure for holding only one of the light guiding component and the imaging assembly. Alternatively, the holding component can be omitted or integrally connected to the auxiliary component.

Besides, as shown in FIG. 2A to FIG. 4, the endoscopic device 1 further includes a heat dissipating tube 18 at least partially and coaxially disposed inside the insertion tube 11. The heat dissipating tube 18 is located at a side of the light emitting component 14 away from the distal end 111 of the insertion tube 11. The auxiliary component 15 is at least partially disposed between heat dissipating tube 18 and the light emitting component 14.

Preferably, the heat dissipating tube 18 can be made of copper, and the insertion tube 11 can be made of stainless steel.

Preferably, as shown in FIG. 2B, the heat dissipating tube 18 is extended to the proximal end 112 of the insertion tube 11.

In order to improve the heat dissipation efficiency, a heat dissipating glue is filled in a space between the heat dissipating tube 18 and the heat dissipating pin 16 and a space between the heat dissipating tube 18 and the auxiliary component 15, so as to form a heat dissipating glue structure 19.

Understandably, in another embodiment, the heat dissipating glue can be filled in only one of the space between the heat dissipating tube and the heat dissipating pin and the space between the heat dissipating tube and the auxiliary component. Alternatively, in another embodiment, the heat dissipating pin can be omitted, and the heat dissipating glue can be filled in the space between the heat dissipating tube and the auxiliary component.

Based on the aforementioned heat dissipating configuration, the heat generated by the light emitting component 14 can be transferred to the heat dissipating tube 18 through the auxiliary component 15, the heat dissipating pin 16 and the heat dissipating glue structure 19 rapidly, so as to achieve a significant temperature control for preventing the light emitting component 14, the image sensor 122 or any other electronic component from being negatively affected by a high temperature and preventing injury of the patient caused by the high temperature.

Furthermore, as shown in FIG. 2A and FIG. 3 to FIG. 4, the endoscopic device 1 further includes a first circuit board C1 electrically connected to the light emitting component 14 and a second circuit board C2 electrically connected to the image sensor 122. The first circuit board C1 and the second circuit board C2 are separated from each other and at least partially received in a channel 153 of the auxiliary component 15 on a bottom side of the auxiliary component 15. A distal portion C11 of the first circuit board C1 is bent relative to the length direction L. A distal portion C21 of the second circuit board C2 protrudes beyond the auxiliary component 15 along the length direction L toward the distal end 111 of the insertion tube 11 and is at least partially received in a channel 173 of the holding component 17 on a bottom side of the middle portion 172 of the holding component 17. The first circuit board C1 and the second circuit board C2 are at least covered by the heat dissipating glue structure 19.

Preferably, the first circuit board C1 and the second circuit board C2 can be flexible printed circuit boards.

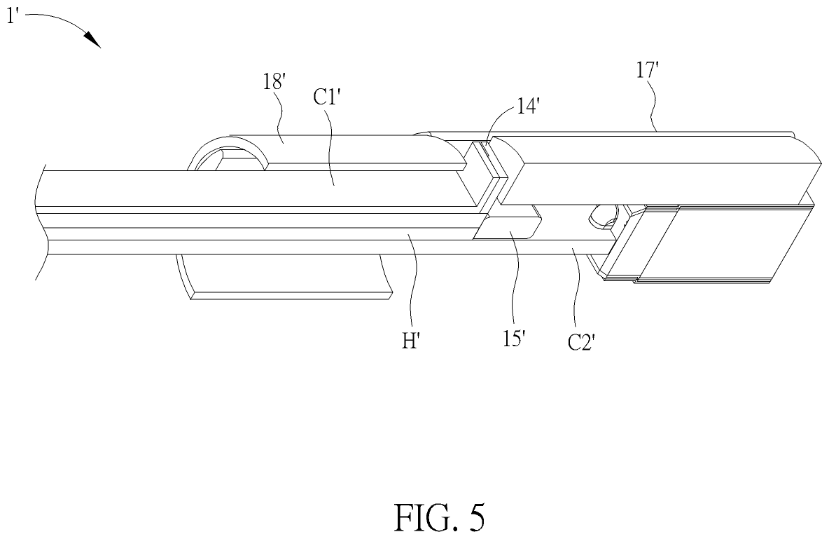

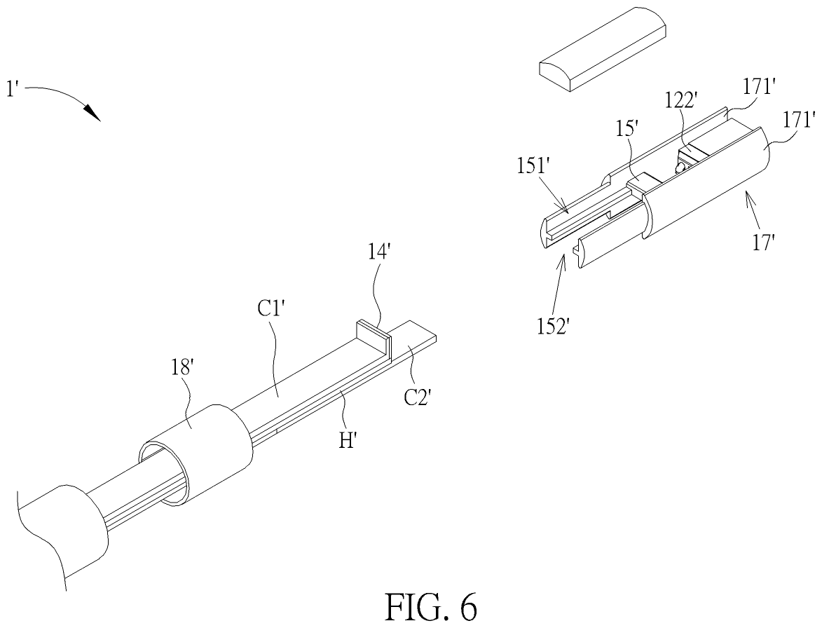

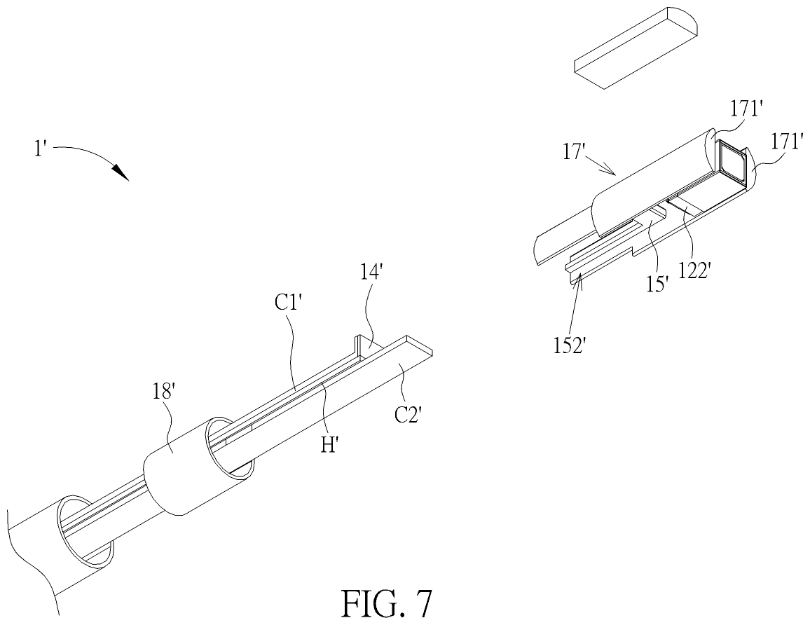

Please refer to FIG. 5 to FIG. 7. FIG. 5 is a partial internal structural diagram of an endoscopic device 1′ according to a second embodiment of the present invention. FIG. 6 and FIG. 7 are partial exploded diagrams of the endoscopic device 1′ at different views according to the second embodiment of the present invention. As shown in FIG. 5 to FIG. 7, different from the first embodiment, in this embodiment, a length of a heat dissipating tube 18′ is shorter than a length of the heat dissipating tube 18 of the first embodiment. Besides, in this embodiment, a top portion of a light emitting component 14′ is not covered by an auxiliary component 15′, and the auxiliary component 15′ includes a first channel 151′ and a second channel 152′ opposite to each other. Furthermore, in this embodiment, a first circuit board C1′ electrically connected to the light emitting component 14′ and a second circuit board C2′ electrically connected to an image sensor 122′ are separated from each other and partially received in the first channel 151′ and the second channel 152′ of the auxiliary component 15′ respectively. Besides, in this embodiment, the endoscopic device 1′ further includes an additional heat dissipating component H′. The additional heat dissipating component H′ is disposed between the first circuit board C1′ and the second circuit board C2′ for improving heat dissipation efficiency, wherein the additional heat dissipating component H′ can be a flat plate structure. Moreover, in this embodiment, a holding component 17′ includes two holding portions 171′ and integrally connected to the auxiliary component 15′, and a heat dissipating tube 18′ is sleeved outside the auxiliary component 15′. Apart from the aforementioned differences, in this embodiment, the endoscopic device 1′ does not include the heat dissipating pin and the heat dissipating glue. Other details of this embodiment are similar to the ones of the first embodiment. Detailed description is omitted herein for simplicity.

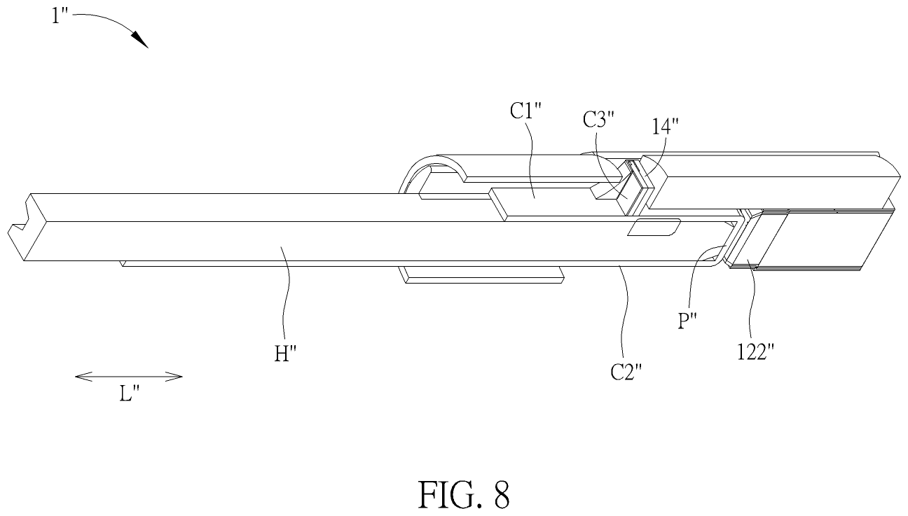

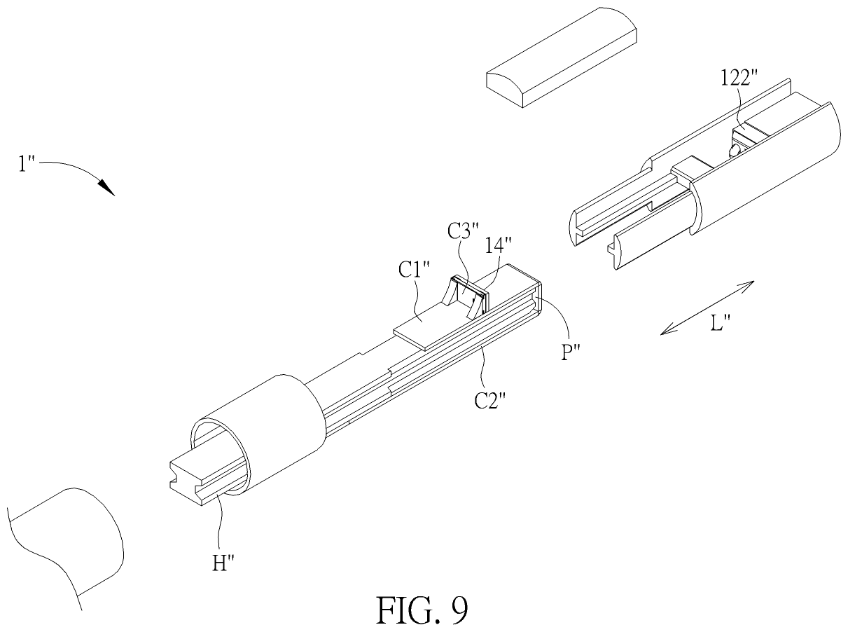

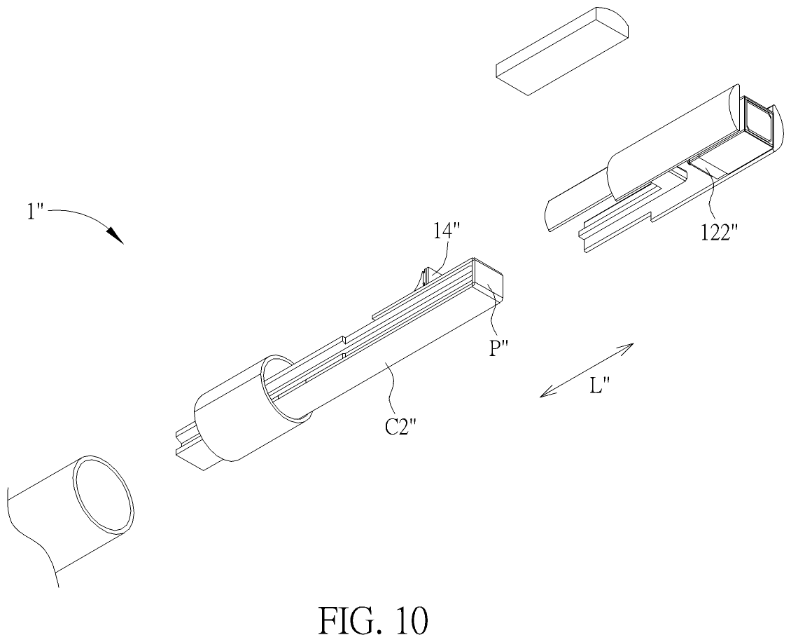

Please refer to FIG. 8 to FIG. 10. FIG. 8 is a partial internal structural diagram of an endoscopic device 1″ according to a third embodiment of the present invention. FIG. 9 and FIG. 10 are partial exploded diagrams of the endoscopic device 1″ at different views according to the third embodiment of the present invention. As shown in FIG. 8 to FIG. 10, different from the second embodiment, in this embodiment, a portion P″ of a second circuit board C2″ electrically connected to an image sensor 122″ is bent relative to a length direction L″, and a first circuit board C1″ is integrated with the second circuit board C2″. Furthermore, the endoscopic device 1″ further includes an auxiliary circuit board C3″ electrically connected between the first circuit board C1″ and the light emitting component 14″ and perpendicular to the first circuit board C1′, so as to provide a flat surface for easy mounting of the light emitting component 14′. Besides, in this embodiment, an additional heat dissipating component H″ is thicker than the additional heat dissipating component H′ of the second embodiment, wherein the additional heat dissipating component H″ of this embodiment can an I-shaped or H-shaped structure, and the additional heat dissipating component H″ abuts against the portion P″ of the second circuit board C2″ electrically connected to the image sensor 122″. Other details of this embodiment are similar to the ones of the second embodiment. Detailed description is omitted herein for simplicity.

In contrast to the prior art, the endoscopic device of the present invention can not only have a compact size and an easy assembling process, but also have an enhanced illumination and an efficient heat dissipation.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. An endoscopic device comprising:
an insertion tube comprising a proximal end and a distal end opposite to the proximal end;
an imaging assembly comprising:
a lens set located adjacent to the distal end of the insertion tube; and
an image sensor located inside the insertion tube and engaged with the lens set;
a light guiding component located adjacent to the distal end of the insertion tube and beside the lens set, the light guiding component comprising an outer end adjacent to the distal end of the insertion tube and an inner end opposite to the outer end, a length of the light guiding component being greater than a length of the imaging assembly; and
a light emitting component located inside the insertion tube and adjacent to the inner end of the light guiding component.

2. The endoscopic device of claim 1, wherein the light emitting component is located at a side of the image sensor away from the distal end of the insertion tube.

3. The endoscopic device of claim 1, wherein the inner end of the light guiding component protrudes beyond the image sensor along a length direction of the insertion tube toward the proximal end of the insertion tube.

4. The endoscopic device of claim 1, further comprising:
an auxiliary component located adjacent to the light emitting component; and
a heat dissipating pin partially embedded inside the auxiliary component.

5. The endoscopic device of claim 4, wherein the light emitting component is partially surrounded by the auxiliary component.

6. The endoscopic device of claim 4, further comprising a holding component detachably engaged with the auxiliary component and for holding the light guiding component or/and the imaging assembly.

7. The endoscopic device of claim 6, wherein a first auxiliary structure is formed on a first end portion of the auxiliary component adjacent to the proximal end of the insertion tube, at least one second auxiliary structure is formed on a second end portion of the auxiliary component opposite to the first end portion of the auxiliary component, the first auxiliary structure is configured to accommodate a portion of the heat dissipating pin exposed out of the auxiliary component, and the at least one second auxiliary structure is configured to engage with the holding component.

8. The endoscopic device of claim 6, wherein the holding component comprises two holding portions, and the light guiding component and the imaging assembly are at least partially located inside a space defined between the two holding portions.

9. The endoscopic device of claim 4, further comprising a heat dissipating tube at least partially and coaxially disposed inside the insertion tube.

10. The endoscopic device of claim 9, wherein a heat dissipating glue is filled in a space between the heat dissipating tube and the heat dissipating pin and/or a space between the heat dissipating tube and the auxiliary component.

11. The endoscopic device of claim 1, further comprising:
a heat dissipating tube at least partially and coaxially disposed inside the insertion tube; and
an auxiliary component at least partially disposed between heat dissipating tube and the light emitting component.

12. The endoscopic device of claim 11, wherein a heat dissipating glue is filled in a space between the heat dissipating tube and the auxiliary component.

13. The endoscopic device of claim 1, further comprising:
a first circuit board electrically connected to the light emitting component;
a second circuit board electrically connected to the image sensor; and
an additional heat dissipating component disposed between the first circuit board and the second circuit board.

14. The endoscopic device of claim 13, wherein the first circuit board is integrated with the second circuit board or separated from the second circuit board.

15. An endoscopic device comprising:
an insertion tube comprising a proximal end and a distal end opposite to the proximal end;
a heat generating component located adjacent to the distal end of the insertion tube; and
a heat dissipating tube at least partially and coaxially disposed inside the insertion tube, the heat dissipating tube being located at a side of the heat generating component away from the distal end of the insertion tube.

16. The endoscope device of claim 15, further comprising:
an auxiliary component at least partially disposed between heat dissipating tube and the heat generating component.

17. The endoscopic device of claim 16, further comprising:
a heat dissipating pin partially embedded inside the auxiliary component.

18. The endoscopic device of claim 17, wherein a heat dissipating glue is filled in a space between the heat dissipating tube and the heat dissipating pin and/or a space between the heat dissipating tube and the auxiliary component.

19. The endoscopic device of claim 17, wherein a first auxiliary structure is formed on a first end portion of the auxiliary component adjacent to the proximal end of the insertion tube, and the first auxiliary structure is configured to accommodate a portion of the heat dissipating pin exposed out of the auxiliary component.

20. The endoscopic device of claim 16, wherein a heat dissipating glue is filled in a space between the heat dissipating tube and the auxiliary component.
